(19) United States
(12) Patent Application Publication
(10) Pub. No.: US 2002/0106715 A1
(43) Pub. Date: Aug. 8, 2002

(54) System and method for collecting data from individual cells

(75) Inventors: Tamir Huberman, Rehovot (IL); Alex Sakin, Jerusalem (IL); Doron Dangour, Lod (IL)

Correspondence Address: DR. MARK FRIEDMAN LTD., C/O Bill Polkinghorn Discovery Dispatch, 9003 Florin Way, Upper Marlboro, MD, 20772, US

(73) Assignee: MEDISEL LTD

(21) Appl. No.: 09/773,583

(22) Filed: Feb. 2, 2001

(51) Int. Cl. C12M 1/34; C12Q 1/24; C12Q 1/04

(52) U.S. Cl. 435/33; 435/30; 435/34; 435/286.5; 435/287.3; 435/288.4; 435/288.7

(57) ABSTRACT

An automated system and method for loading individual cells into individual discrete locations. The system includes a cell carrier grid, a cell carrier grid holder, a vacuum source, a liquid reservoir and a loading device facilitating communication between the above components. Application of vacuum via a port causes cells to move into individual discrete locations. The method includes the steps of placing the grid holder into a loading device, automatically filling a space in the grid holder with a liquid, automatically adding to an upper surface of the grid and automatically applying a force to the cells in so that individual cells enter at least some of the individual discrete locations. Further disclosed is an automated system for collection of data from cells further including an electro-optical scanner capable of illuminating the cells and collecting at least a portion of photons therefrom and a computerized control mechanism further controlling same. Further disclosed is an automated method of collection of data from cells comprising the additional steps of illuminating the cells by means of an electro-optical scanner and collecting at least a portion of photons therefrom and co-ordinating actions of the electro-optical scanner by means of a computerized control mechanism.

Further disclosed is an article of manufacture including a cell carrier grid, a cell carrier grid holder, a vacuum source a liquid reservoir a loading device, an electro-optical scanner and a computerized control mechanism designed and configured to co-ordinate actions the above components, said computerized control mechanism operable with a graphical user interface.

### FIELD AND BACKGROUND OF THE INVENTION

The present invention relates to an improved system and method for collecting data from individual cells and, more particularly, to automation of the process of causing cells to reside in individual discrete locations and of addition of that automated process to collection of data from cells residing in individual discrete locations in a cell carrier grid. The present invention further relates to an article of manufacture which includes an electro-optical scanner, cell carrier grids and a loading device for same. The present invention further relates to systems and methods which allow recovery of specific cells residing in individual discrete locations based upon data collected therefrom.

Because of the complex nature of biological systems, it is often desirable to conduct analyses on a specific sample to compare to normative values. For example, liver enzyme levels from a specific patient compared to normative values for the same enzyme may be used to diagnose diabetes.

Further, it is often desirable to assay cells taken from a specific organ or tissue in order to diagnose a condition in a patient. In some cases, a sample may contain a physiologically mixed population of cells, only a portion of which is to be analyzed. Machines such as a fluorescence activated cell sorter (FACS) were designed, in part, to overcome this problem. However, a FACS machine cannot reassay individual cells after sorting. This limitation precludes both kinetic studies of individual cells and recovery of individual cells after assay based upon assay results.

Therefore, a number of prior art devices were patented by Weinraub et al. to address some of these issues. (U.S. Pat. Nos. 4,729,949; 5,272,081; 5,310,674; and 5,506,141)

U.S. Pat. No. 4,729,949 teaches methods and apparatus for performing analyses on individual living cells. According to the teachings of this patent, individual cells are forced into holes in a carrier grid so that each of the cells may be individually assayed and re-assayed. The teachings of this include instrument means for observing or measuring one or more properties of individual cells and control means for controlling the relative locations of the instrument means and the carrier grid so that the instrument means is directed to a particular cell to observe or measure the one or more properties the cell. The instrument means may include optical scanning means for determining optical properties of the living cells according to the teachings of this patent. However, teachings of this patent do not disclose such a scanning means. Further, the teachings of this patent do not include an optical shutter, such a shutter greatly increasing the range of measurement achievable with a scanning means.

U.S. Pat. No. 5,310,674 is similar except that it teaches an ordered array of holes of two different sizes so that sorting of cells by size into two subpopulations is theoretically feasible Like U.S. Pat. No. 4,729,949, the teachings of this patent do not include an optical shutter, a limitation that severely limits the range of measurement achievable according to the teachings of this patent.

U.S. Pat. No. 5, 272,081 teaches identification and subculture of a selected subgroup of cells residing in a grid of the type taught in U.S. Pat. Nos. 4,729,949. 5,506,141 is similar to U.S. Pat. No. 4,729,949 except that it teaches that “the positions on the carrier of the holes are identifiable.” The same inherent drawbacks are present in the teachings of these patent.

U.S. Pat. No. 4,772,540 to Deutsch et al. teaches a method of manufacture for a rigid grid resistant to mechanical distortion. Despite the added strength, grids produced according to the teachings of Deutsch do not hold cells in a single focal plane. A scanning means is not disclosed in this patent, although cells are presumably scanned during use of the disclosed invention.

Preparation of cells for assay according to teachings of patents cited hereinabove is a manual process which is both time consuming and requires employment of trained personnel. Similarly, analysis of data in these prior art patents is a process which requires attention of an operator of the patented methods and devices. Further, direct collection of individual cells based upon results of an assay performed thereuopon is not taught by the prior art.

There is thus a widely recognized need for, and it would be highly advantageous to have, an improved system and method for collecting data from individual cells devoid of the above limitation.

### SUMMARY OF THE INVENTION

According to one aspect of the present invention there is provided an automated system for loading individual cells from a population of cells in suspension into individual discrete locations within an array of individual discrete locations located in a cell carrier grid contained in a cell carrier grid holder. The system comprises: (a) the cell carrier grid, the grid held in the cell carrier grid holder such that a lower surface of the grid is in communication with a space within the holder; (b) the cell carrier grid holder, (c) a vacuum source connectable to the port; (d) at least one liquid reservoir for bringing at least one liquid into contact with the individual cells from a population of cells in suspension while the individual cells reside in the individual discrete locations; and (e) a loading device facilitating communication between the grid holder containing the grid, the vacuum source, the population of cells in suspension, and the at least one liquid reservoir. Application of vacuum via the port causes the individual cells from the population of cells in suspension to move into the individual discrete locations. The at least one liquid may be applied to the individual cells from a location selected from the group consisting of the space and an upper surface of the cell carrier grid. The grid holder holder comprises: (i) the space in communication with the lower surface of the grid; (ii) at least one port for introduction of a liquid into the space; and (iii) the at least one port further serving for removal of the liquid from the space.

According to another aspect of the present invention there is provided an automated method for loading individual cells from a population of cells in suspension into individual discrete locations within an array of individual discrete locations located in a cell carrier grid contained in a cell carrier grid holder. The method comprises the steps of: (a) placing the grid holder into a loading device; (b) automatically filling a space in the cell carrier grid holder with a liquid such that the liquid fills the individual discrete locations; (c) automatically adding a portion of the cells in suspension to an upper surface of the grid; (d) automatically applying a force to the portion of the cells in suspension so that individual cells enter at least some of the individual discrete locations.

According to yet another aspect of the present invention there is provided an automated system useful for collection of data from a plurality of individual cells belonging to a population of cells in suspension. The system comprises: (a) a cell carrier grid including a plurality of individual discrete locations arranged in an array such that each of the individual discrete locations is capable of engaging and retaining one of the individual cells, the grid held in a grid holder such that a lower surface of the grid is in communication with a space within the holder; (b) the cell carrier grid holder (c) a vacuum source connectable to the port; (d) at least one liquid reservoir for bringing at least one liquid into contact with the individual cells from the population of cells in suspension while the individual cells reside in the individual discrete locations; (e) a loading device facilitating communication between the grid holder containing the grid, the vacuum source, the population of cells in suspension, and the at least one liquid reservoir;

wherein application of vacuum via the port causes the individual cells from the population of cells in suspension to move into the individual discrete locations; (f) an electro-optical scanner capable of illuminating the individual cells residing in the individual discrete locations and collecting at least a portion of photons emanating from the individual cells residing in the individual discrete locations and (g) a computerized control mechanism designed and configured to co-ordinate actions of the cell carerier grid holder, the vacuum source, the at least one population of cells in suspension, the at least one liquid reservoir, the loading device and the electro-optical scanner. The at least one liquid may be applied to the individual cells from a location selected from the group consisting of the space and an upper surface of the cell carrier grid. The grid holder holder comprises: (i) the space in communication with the lower surface of the grid; (ii) at least one port for introduction of a liquid into the space; and (iii) the at least one port further serving for removal of the liquid from the space.

According to still another aspect of the present invention there is provided an automated method of collection of data from a plurality of individual cells belonging to a population of cells in suspension. The method comprises the steps of: (a) providing a cell carrier grid including a plurality of individual discrete locations arranged in an array such that each of the individual discrete locations is capable of engaging and retaining one of the individual cells, and holding the grid held in a grid holder such that a lower surface of the grid is in communication with a space within the holder; (b) allowing at least one liquid to enter and leave the space in the grid holder via at least one port; (c) causing the individual cells from the population of cells in suspension to move into the individual discrete locations by means of a vacuum source connectable to the port; (d) supplying the population of cells in suspension; (e) allowing communication between the at least one liquid in at least one liquid reservoir reservoir and the individual cells from the population of cells in suspension while the individual cells reside in the individual discrete locations wherein the at least one liquid may communicate with the individual cells from a location selected from the group consisting of the space and an upper surface of the cell carrier grid; and (f) employing a loading device to facilitate communication between the grid holder containing the grid, the vacuum source, the population of cells in suspension, and the at least one liquid reservoir; (g) illuminating the individual cells residing in the individual discrete locations and collecting at least a portion of photons emanating from the individual cells residing in the individual discrete locations by means of an electro-optical scanner; and (h) co-ordinating actions of the cell carerier grid holder, the vacuum source, the population of cells in suspension, the at least one liquid reservoir, the loading device and the electro-optical scanner by means of a computerized control mechanism.

According to an additional aspect of the present invention there is provided an article of manufacture useful for collection of data from a plurality of individual cells belonging to a population of cells in suspension in a clinical setting. The article of manufacture comprises: (a) a cell carrier grid including a plurality of individual discrete locations arranged in an array such that each of the individual discrete locations is capable of engaging and retaining one of the individual cells, the grid held in a grid holder such that a lower surface of the grid is in communication with a space within the holder; (b)the cell carrier grid holder; (c) a vacuum source connectable to the port; (d) at least one liquid reservoir for bringing at least one liquid into contact with the individual cells from the population of cells in suspension while the individual cells reside in the individual discrete locations; (e) a loading device facilitating communication between the grid holder containing the grid, the vacuum source, the population of cells in suspension, and the at least one liquid reservoir; (f) an electro-optical scanner capable of illuminating the individual cells residing in the individual discrete locations and collecting at least a portion of photons emanating from the individual cells residing in the individual discrete locations; and (g) a computerized control mechanism designed and configured to co-ordinate actions of the cell carerier grid holder, the vacuum source, the population of cells in suspension, the at least one liquid reservoir, the loading device and the electro-optical scanner, the computerized control mechanism operable with a graphical user interface. Application of vacuum via the port causes the individual cells from the population of cells in suspension to move into the individual discrete locations. The at least one liquid may be applied to the individual cells from a location selected from the group consisting of the space and an upper surface of the cell carrier grid. The grid holder holder comprises: (i) the space in communication with the lower surface of the grid; (ii) at least one port for introduction of a liquid into the space; and (iii) the at least one port further serving for removal of the liquid from the space.

According to yet additional aspect of the present invention there is provided an improved electro-optical scanner capable of individually collecting data from a plurality of individual cells residing in predefined locations. The scanner comprises: (a) an optical unit, the optical unit comprises a camera, a light source, a photomultiplier, an optical shutter, and at least one optical filter; (b) a cell carrier grid, the grid comprises an array of discrete locations, each of the discrete locations capable of engaging and retaining a single living cell; (c) a scanning unit capable of exposing the discrete locations to light from the light source; (d) a cell manipulation device selected from the group consisting of a micropipette, a needle, and an electrode and (e) a control unit, the control unit comprises a computer designed and configured for co-ordinating actions of the optical unit, the cell carrier grid, the scanning unit and the cell manipulation device.

According to still additional aspect of the present invention there is provided a method of collecting data from individual cells belonging to a plurality of individual cells residing in predefined locations by means of an improved electro-optical scanner. The method comprises the steps of: (a) causing individual cells from the plurality of individual cells to be engaged and retained in discrete locations belonging to an array of discrete locations in a cell carrier grid; (b) exposing the discrete locations to light from a light source by employing a scanning unit; (c) generating the data from an an optical unit, the optical unit comprises a camera, the light source, a photomultiplier, an optical shutter, and at least one optical filter; (d) manipulating individual cells from the plurality of individual cells with a cell manipulation device selected from the group consisting of a micropipette, a needle, and an electrode; and (e) co-ordinating actions of the optical unit, the cell carrier grid and the cell manipulation device and the scanning unit from a control unit, the control unit comprises a computer.

According to further features in preferred embodiments of the invention described below, the grid holder is constructed of at least one material selected from the group consisting of Lucite, plastic, glass, silicon and metal

According to still further features in the described preferred embodiments the invention further comprises at least one robotic mechanism.

According to still further features in the described preferred embodiments the at least one robotic mechanism is designed and configured for performing at least one function selected from the group consisting of: (i) placing the grid holder into the loading device; (ii) removing the grid holder from the loading device (iii) transferring the grid holder to a scanning assay device; and (iv) removing the grid holder from the scanning assay device.

According to still further features in the described preferred embodiments the robotic mechanism includes at least one item selected from the group consisting of at least one robotic arm, at least one conveyor belt, at least one pneumatic tube, at least one piston and at least one rotating plate.

According to still further features in the described preferred embodiments the port comprises a first port serving for introduction of a liquid into the space and a second port serving for removal of the liquid from the space.

According to still further features in the described preferred embodiments the system further comprises a computerized control mechanism designed and configured to co-ordinate the actions of the vacuum source, the at least one population of cells in suspension, the loading device and the at least one liquid reservoir.

According to still further features in the described preferred embodiments the system further comprises a computerized control mechanism designed and configured to co-ordinate the actions of the vacuum source, the at least one population of cells in suspension, the loading device, the at least one liquid reservoir, and the at least one robotic mechanism.

According to still further features in the described preferred embodiments the at least one reagent contained within the at least one liquid is capable of imparting a measurable degree of fluorescence to the cells in the suspension at at least one wavelength.

According to still further features in the described preferred embodiments the at least one reagent capable of imparting a measurable degree of fluorescence is selected from the group consisting of: (i) a substance that differentially stains living cells; (ii) a precursor of a fluorescent substance that differentially stains living cells; (iii) a fluorophore that stains nucleic acids; and (iv) a flourescently labeled antibody.

According to still further features in the described preferred embodiments the method further comprises the step of bringing the cells in the individual discrete locations into contact with at least one liquid.

According to still further features in the described preferred embodiments the step of placing the grid holder into a loading device is further automated.

According to still further features in the described preferred embodiments the step of placing the grid holder into the loading device is accomplished with the aid of at least one robotic mechanism.

According to still further features in the described preferred embodiments the method further comprises at least one additional step selected from the group consisting of: (i) removing the grid holder from the loading device; (ii) transferring the grid holder to a scanning assay device; and (iii) removing the grid holder from the scanning assay device; is performed by at least one robotic mechanism designed and configured for performing the at least one additional step.

According to still further features in the described preferred embodiments wherein the steps of automatically filling a space, and automatically applying a force are accomplished by causing a liquid to flow through at least one port in the grid holder.

According to still further features in the described preferred embodiments causing the liquid to flow includes causing the liquid to flow through: (i) a first port serving for introduction of the liquid into the space; and (ii) a second port serving for removal of the liquid from the space.

According to still further features in the described preferred embodiments the steps of automatically filling a space, automatically adding a portion of the cells, and automatically applying a force are co-ordinated by a computerized control mechanism.

According to still further features in the described preferred embodiments the electro-optical scanner comprises: (i) an optical unit, the optical unit comprises a camera, a light source, a photomultiplier, an optical shutter, and at least one optical filter; and (ii) a scanning unit capable of exposing the discrete locations to light from the light source. The optical unit and components thereof and the scanning unit are controlled by the computerized control mechanism

According to still further features in the described preferred embodiments the electro-optical scanner further comprises a cell manipulation device selected from the group consisting of a micropipette, a needle, and an electrode and the control unit further co-ordinates actions of the cell manipulation device.

According to still further features in the described preferred embodiments the micropipette is capable of an action selected from the group consisting of removing at least a portion of an organelle from an individual cell, removing at least a portion of the individual cell's cytoplasm, and removing the individual cell from one of the discrete locations.

According to still further features in the described preferred embodiments the needle is capable of an action selected from the group consisting of injecting a substance into an individual cell residing in the discrete location and extracting a substance from an individual cell residing in the discrete location.

According to still further features in the described preferred embodiments the electrode is capable of an action selected from the group consisting of applying an electric current to an individual cell residing in the discrete location, measuring a potential difference across a membrane of an individual cell residing in the discrete location, and creating a potential difference across a membrane of an individual cell residing in the discrete location.

According to still further features in the described preferred embodiments the electro-optical scanner capable of collecting at least a portion of photons emanating from the individual cells residing in the individual discrete locations is further capable of gathering polarization data pertaining to the photons.

According to still further features in the described preferred embodiments the polarization data is useful in making a medical diagnosis.

According to still further features in the described preferred embodiments the method comprises the additional step of providing at least one robotic mechanism.

According to still further features in the described preferred embodiments the at least one robotic mechanism performs at least one function selected from the group consisting of: (i) placing the grid holder into the loading device; (ii) removing the grid holder from the loading device (iii) transferring the grid holder to a scanning assay device; and (iv) removing the grid holder from the scanning assay device.

According to still further features in the described preferred embodiments the method comprises the additional step of including within the electro-optical scanner a cell manipulation device selected from the group consisting of a micropipette, a needle, and an electrode and the control unit further co-ordinates actions of the cell manipulation device.

According to still further features in the described preferred embodiments the step of illuminating the individual cells residing in the individual discrete locations and collecting at least a portion of photons emanating from the individual cells residing in the individual discrete locations further includes gathering polarization data pertaining to the photons.

According to still further features in the described preferred embodiments the article of manufacture further comprises instructions for performing specific analyses therewith, the instructions reducing the need for calibration thereof.

According to still further features in the described preferred embodiments the article of manufacture further comprises a cell manipulation device.

The present invention successfully addresses the shortcomings of the presently known configurations by providing an improved system and method for collecting data from individual cells and, more particularly, to automation of the process of causing cells to reside in individual discrete locations and of addition of that automated process to collection of data from cells residing in individual discrete locations in a cell carrier grid. The present invention further relates to an article of manufacture which includes an electro-optical scanner, cell carrier grids and a loading device for same. The present invention further relates to systems and methods which allow recovery of specific cells residing in individual discrete locations based upon data collected therefrom. The present invention successfully addresses further shortcomings of previously known configurations by incorporating an optical shutter into an electro-optical scanner in order to prevent bleaching and biological damage.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic representation of essential components of one embodiment of an improved electro-optical scanner according to some aspects of the present invention;

FIG. 2 is a diagrammatic representation of a physical arrangement of parts in an improved electro-optical scanner as shown in FIG. 1;

FIG. 3 is a;

FIG. 4 is a cross sectional view of an automated system for loading individual cells from a population of cells in suspensi and for transferring the grid holder to an assay device according to the present invention;

FIG. 5 is a flow diagram of method steps according to the present invention;

FIGS. 6a-i illustrate different embodiments of cell manipulation devices according to the present invention;

FIGS. 7a-e illustrate different embodiments of robotic mechanisms according to the present invention.

### DESCRIPTION OF THE PREFERRED EMBODIMENTS

The present invention is of an improved system and method for collecting data from individual cells In particular, the present invention relates to automation of the process of causing cells to reside in individual discrete locations and of addition of that automated process to collection of data from cells residing in individual discrete locations in a cell carrier grid. The present invention further relates to an article of manufacture which includes an electro-optical scanner, cell carrier grids and a loading device for same. The present invention further relates to systems and methods which allow recovery of specific cells residing in individual discrete locations based upon data collected therefrom.

The principles and operation of systems, method and articles of manufacture according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting. Specifically, embodiments of the present invention will often include commercially available components. One ordinarily skilled in the art will be capable of selecting and assembling such components. Details of specific commercially available components provided herein are provided as non-limiting examples. Substitution of analagous components can easily be effected without substantially altering the invention.

Referring now to the drawings, FIG. 4 illustrates an automated system 70 for loading individual cells from a population of cells 81 in suspension into individual discrete locations 77 within an array of individual discrete locations 77 located in a cell carrier grid 5 contained in a cell carrier grid holder 71.

Grid holder 71 may be constructed of any material including, but not limited to, Lucite, plastic, glass, silicon and metal. System 70 includes cell carrier grid 5 and grid holder 71 holding grid 5 such that a lower surface 80 of grid 5 is in communication with a space 72 within holder 71. System 70 further includes a vacuum source 74 connectable to a port 73b. System 70 further includes at least one liquid reservoir 75 for bringing at least one liquid into contact with the individual cells from a population of cells 81 in suspension while the individual cells reside in the individual discrete locations 77. System 70 further includes a loading device 76 facilitating communication between grid holder 71 containing grid 5, vacuum source 74, population of cells 81 in suspension, and liquid reservoir 75. Application of vacuum via port (two ports 73a and 73b are pictured) causes the individual cells from the population of cells 81 in suspension to move into the individual discrete locations 77. The at least one liquid may be applied to the individual cells from a location selected from the group consisting of space 72 and an upper surface 82 of grid 5. Holder 71 includes space 72 in communication with lower surface 80 of grid 5, at least one port 73 (two ports 73a and 73b are pictured) for introduction of a liquid into space 72 and for removal of the liquid from space 72.

According to preferred embodiments of the invention port 73 includes first port 73a serving for introduction of a liquid into space 72 and second port 73b serving for removal of the liquid from space 72. Liquid reservoir 75 and vacuum source 74 may be connected to ports 73a and 73b by connecting means 79 a and 79b respectively. Connecting means 79 may be, for example a tube, a pipe, a sleeve, a gasket or a flange.

System 70 may further include at least one robotic mechanism 78. According to various embodiments of the invention, robotic mechanism 78 is designed and configured for performing at least one function. The at least one function may include, but is not limited to, the following functions:

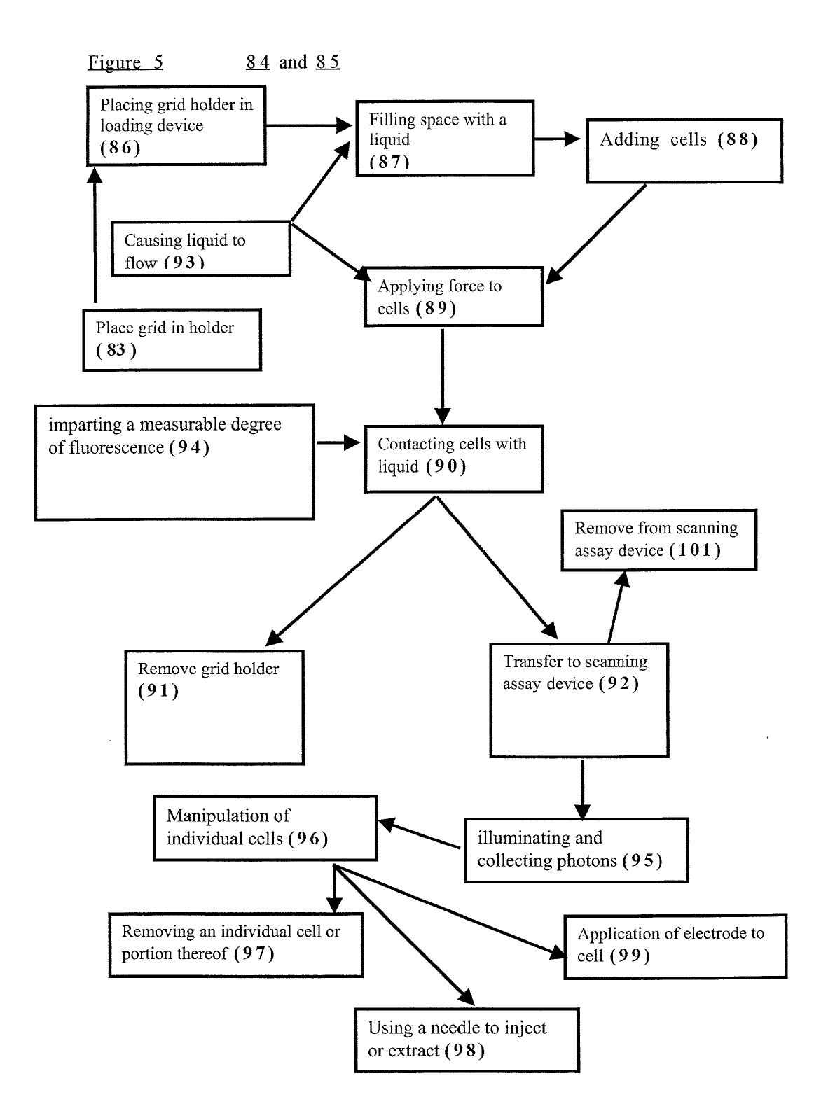

Placing 86 (FIG. 5) grid holder 71 into loading device 76;

Removing 91 grid holder 71 from loading device 76;

Transferring 92 grid holder 71 to a scanning assay device 25; and

Removing 101 grid holder 71 from the scanning assay device 25.

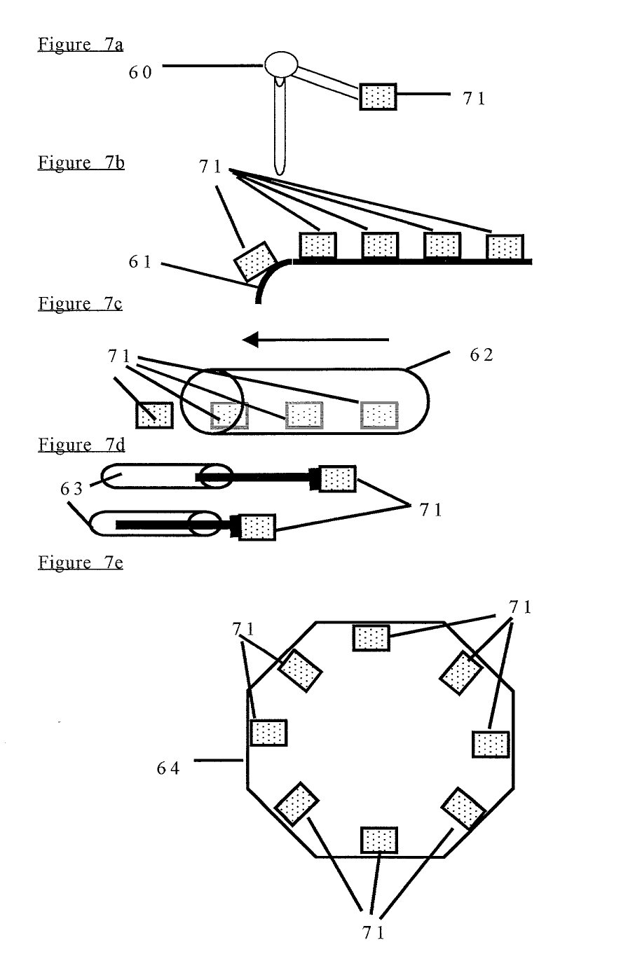

Robotic mechanism 78 may include, for example, at least one robotic arm 60 (FIG. 7a), at least one conveyor belt 61 (FIG. 7b), at least one pneumatic tube 62 (FIG. 7c; arrow indicates direction of flow), at least one piston 63 (FIG. 7d), at least one rotating plate 64 (FIG. 7e) or any combination thereof.

Preferably, system 70 further includes a computerized control mechanism 15 designed and configured to co-ordinate the actions of vacuum source 74, population of cells 81 in suspension, the loading device 76 and liquid reservoir 75. According to additional embodiments of the invention, computerized control mechanism 15 further controls robotic mechanism 78.

Preferably, liquid reservoir 75 contains at least one reagent within the at least one liquid contained therein which is capable of imparting a measurable degree of fluorescence to cells 81 at at least one wavelength.

The at least one reagent capable of imparting a measurable degree of fluorescence may be, for example a substance that differentially stains living cells. Alternately or additionally the at least one reagent capable of imparting a measurable degree of fluorescence may be a precursor of a fluorescent substance that differentially stains living cells. Alternately or additionally the at least one reagent capable of imparting a measurable degree of fluorescence may be a fluorophore that stains nucleic acids. Alternately or additionally the at least one reagent capable of imparting a measurable degree of fluorescence may be a flourescently labeled antibody.

The present invention is further embodied by an automated method 85 (FIG. 5) for loading individual cells from population of cells 81 in suspension into individual discrete locations 77 within an array of individual discrete locations 77 located in cell carrier grid 5 contained in cell carrier grid holder 71. Method 85 includes the steps of placing 86 grid holder 5 into loading device 76, automatically filling 87 space 72 in grid holder 71 with a liquid such that the liquid fills individual discrete locations 77, automatically adding 88 a portion of cells 81 an upper surface 82 of grid 5 and automatically applying 89 a force to cells 81 so that individual cells enter at least some of individual discrete locations 77. Preferably the steps of automatically filling 87 space 72, automatically adding 88 a portion of cells 81, and automatically applying a force 89 are co-ordinated by computerized control mechanism 15. According to some preferred embodiments, the step of placing 86 grid holder 5 into loading device 76 is further automated. This automation may be accomplished, for example, with the aid of at least one robotic mechanism 78.

The present invention is further embodied by an automated system 65 useful for collection of data from a plurality of individual cells belonging to a population of cells 81 in suspension. The system includes grid 5 including a plurality of individual discrete locations 77 arranged in an array such that each of locations 77 is capable of engaging and retaining one of the individual cells. Grid 5 is held in grid holder 71 such that lower surface 80 of grid 5 is in communication with space 72 in holder 71. System 65 further includes holder 71 and vacuum source 74 connectable to port 73 and at least one liquid reservoir 75 for bringing at least one liquid into contact with cells individual cells reside in individual discrete locations 77. System 65 further includes a loading device 76 facilitating communication between grid holder 71 containing grid 5, vacuum source 74, population of cells 81 in suspension, and at least one liquid reservoir 75. During use of system 65, application of vacuum via port 73 causes the individual cells from the population of cells 81 in suspension to move into individual discrete locations 77. System 65 further includes an electro-optical scanner 25 capable of illuminating cells 81 residing in locations 77 and collecting at least a portion of photons emanating therefrom.

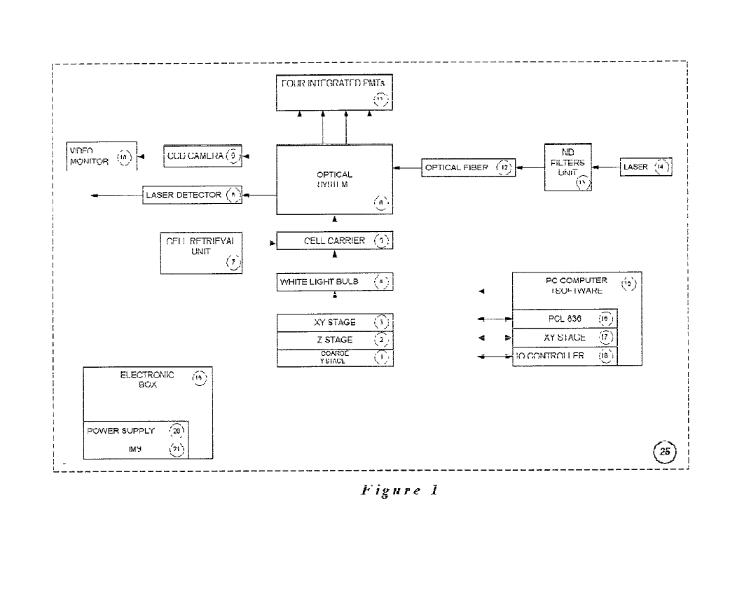

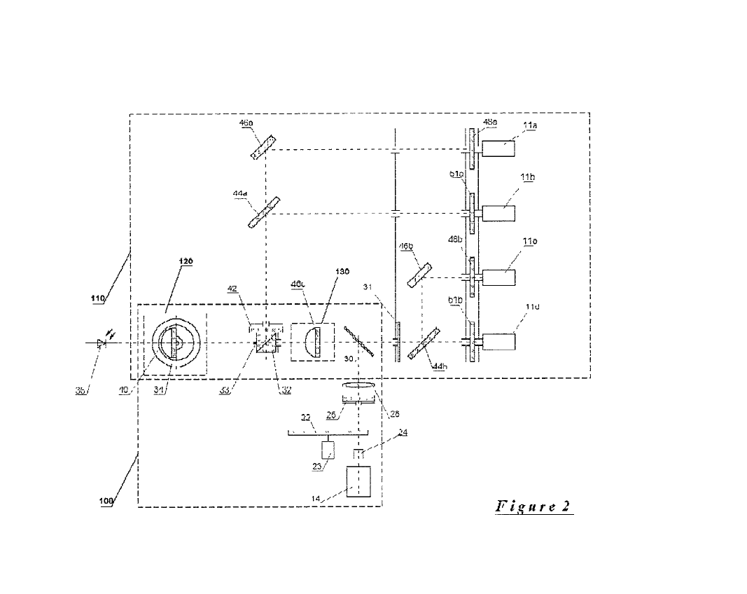

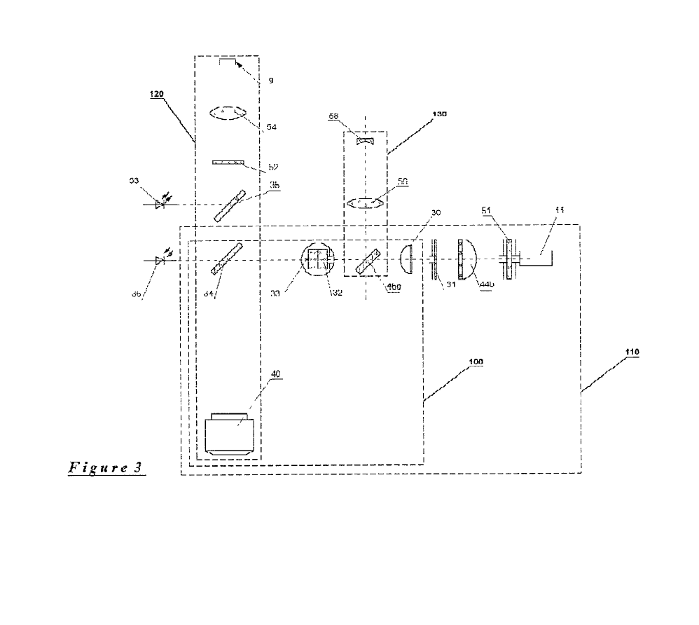

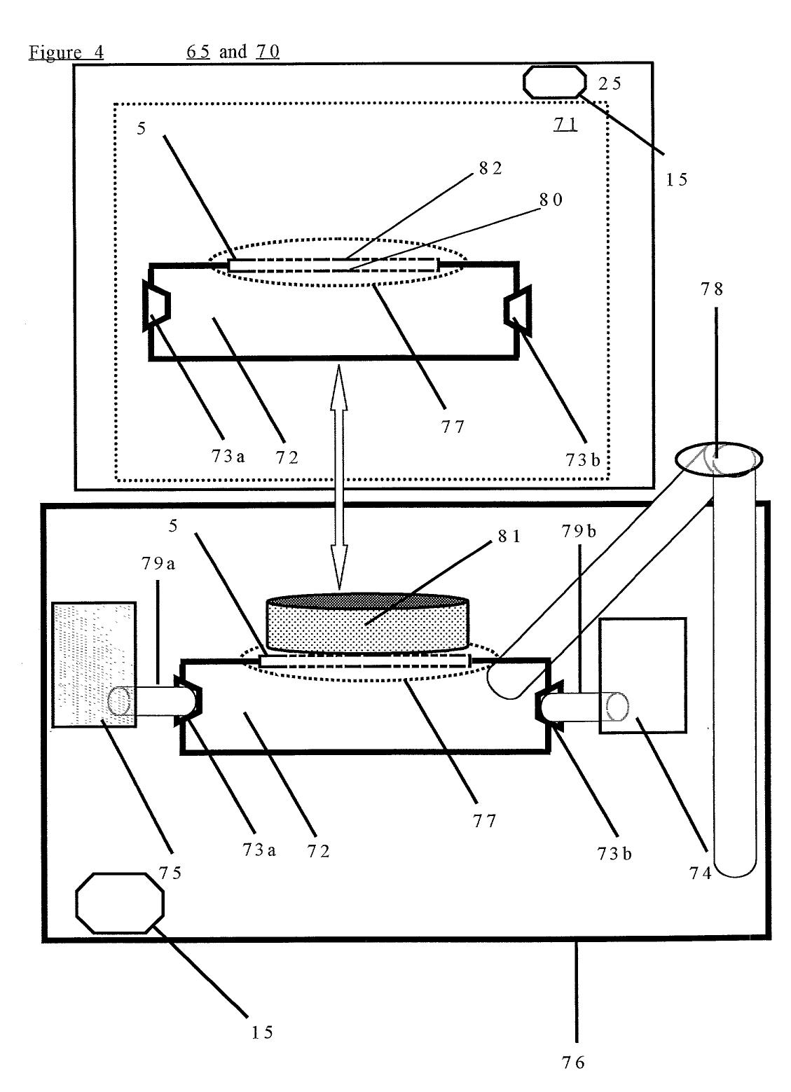

FIGS. 1, 2 and 3 illustrate an improved electro-optical scanner 50 according to the present invention. Scanner 50 is capable of individually collecting data from a plurality of individual cells residing in predefined locations 77. Scanner 50 includes an optical unit 6. Components of optical unit 6 include, but are not necessarily limited to, a camera (e.g. CCD camera 9, a light source (e.g. laser 14), a photomultiplier (e.g. 4 integrated photomultipliers (PMTs) 11), an optical shutter (Q switch 24; FIG. 2), and at least one optical filter 22 (FIG. 2). Scanner 50 may further include a cell carrier grid 5 including an array of discrete locations 77, each of the discrete locations capable of engaging and retaining a single living cell. One ordinarily skilled in the art will be able to incorporate grids, such as those disclosed in patents cited in the background section hereinabove, or other grids which may become available as a result of improvements in the art, for use with scanner 50. Scanner 50 further includes a scanning unit (1, 2, and 3) capable of exposing discrete locations 77 to light from light source 14.

The XY table driver card 17 of controller 15 controls the XY stage 3. The Z 2 stage (Newport stage M-426, Low profile crossed roller bearing translation stage, and CMA-12PP 12.5 mm travel open loop stepper CMA actuator), is controlled by a driver (IMS 21 483, Intelligent Motion Systems, New-Jersey, USA, located in the electronic box 19). The coarse Y stage 1 (MICOS, MT-65 measuring stage Umkirch, Germany), is also controlled by an IMS 483 driver. Software installed in 15 controls the movement of all the stages. Specific manufacturer and model designations are provided not to limit the scope of the invention, but rather to aid one skilled in the art in practice thereof.

A control unit 15 in the form of a computer is provided for co-ordinating actions of optical unit 6, cell carrier 5 and scanning unit 1, 2, and 3. Design and configuration of this computer include an easy to use graphical user interface (GUI), networking capabilities for data storage and transfer and capacity to perform different actions on different samples according instructions received from an operator thereof.

The invention is further embodied by a method 84 of collecting data from individual cells belonging to a plurality of individual cells residing in predefined locations by means of improved electro-optical scanner 50. Method 84 includes the steps of causing individual cells to be engaged and retained in discrete locations in a cell carrier 5, exposing the discrete locations to light 95 from a light source 14 by employing scanning unit (1, 2, and 3) and generating the data from optical unit 6 including camera 9, light source 14, a photomultiplier 11(In the pictured embodiment, Four integrated PMTs 11 (Hamamatsu, Shizuka-Ken, Japan) are used for intensity count, two separate wavelengths) optical shutter 24, and at least one optical filter 22. Light source 14 may be switched off, turned on, and controlled with respect to intensity by IO controller 18. Coordinating of actions of the optical unit 6, the cell carrier 5 and the scanning unit (1, 2 and 3) is by a control unit 15 including a computer.

Control unit 15 may include, for example, three PC embedded electronic cards to control scanner 50. The first card may be, for example, a PCL-836 16 (Multifunction Counter/Timer and digital 10 card, Advantech, Taipei, Taiwan). The second card may be, for example, an XY stage driver card 17 (ISA bus, digital controller, with an onboard linear amplification for two axes, PI (Physik Instrumente, Waldbronn, Germany). The third card may be, for example, an IO controller 18 that controls all data acquisition and control (Medis Technologies, Yehud, Israel).

Cell Carrier 5 is placed on the XY 3, Z 2, and coarse Y 1 stages, after it has been loaded with cells as described hereinabove. Mechanical coarse Y stage 1 quickly centers grid 5 with respect to camera 9 which may be, for example a B/W ⅓″ CCD Camera ( Sony, Japan) or a color CCD or any other device which enables viewing grid 5 for the purpose of orientation.

Viewing the cell locations 77 on the video screen 10 defines for computer 15 the location of each individual hole. Screen 10 may also be used in cell retrieval monitoring. The operator selects a field of interest and scans the grid using electronically controlled stages 2 and 3. For each specific location 77 parameters are measured. These parameters may include, but are not limited to, time of measurement, intensity data (as measured by PMTs 11), and cell location. Measured parameters are stored in a data file on controller 15. According to preferred embodiments of the invention, the data is continuously displayed in real-time in the course of the scan. All stored data can be read and analyzed by numerous algorithms present on computer 15, or on a remote computer. Laser 14 may be, for example, a solid-state diode pumped laser LCS-DTL-362 (Laser Compact, Moscow, Russia; wavelength =473 nm; P≦10 mW) or a He-Cd laser (Liconix Inc Santa-Clara, Calif., USA; wavelength =442 nm) or an Argon laser (Uniphase Inc. Manteca, Calif., USA; wavelength =488 mn) according to user requirements. In some cases, scanner 50 will include multiple lasers 14 which may be specified by a user thereof according to a specific assay being performed. Power supplies 20 are located in electronic box 19 and supply all the power to the system. Alternately or additionally, some components may be powered by direct connection to a standard electric outlet.

The laser enters the optical system by means of a fiber optic cable 12 (e.g. Oz Optics, Ontario, Canada) which transfers the laser beam from laser 14 into optical unit 6. The laser detector 8 may be, for example, an Optic-Hybrid silicon detector (Centronic, New Addington, England, OSI 5-V-10M/10K). Detector 8 monitors the laser intensity and maintains it constant by a closed loop with the ND filter 22 driven by the ND motor 23 (Maxon DC motor, Sachseln, Switzerland).

According to preferred embodiments of the invention, scanner 50 further includes a cell manipulation device 7. Cell manipulation device 7 may be, for example a micropipette 37, needle 38, or electrode 39 capable of performing actions described hereinbelow. According to these embodiments, control unit 15 further co-ordinates actions of cell manipulation device 7.

Cell manipulation device 7, in the form of cell retrieval unit 7 (Eppendorf, Cologne, Germany) is located near the XY stage 3. After a specific cell location is determined by scanning of grid 5, a specific cell is retrieved for later use (PCR, cloning etc'). Designation of which cells to retrieve may be either by an operator of scanner 50, or alternately and preferably, by controller 15 based upon an automated analysis of data.

Optical system 6 provides an optical signal for repeatable real time measurement of fluorescent-polarization in living cells. Prior to measurement the system establishes the grid orientation as detailed hereinabove. This establishes an address for each discrete location in cell carrier 5.

Laser radiation wavelengths of, for example, 473 nm, 442 nm or 488 nm are selectable by an operator of scanner 50. A polarizer 32 divides the fluorescent beam (I), emanating from the cell, to two beams (I₁ and I₂), which are further polarized to two separate orthogonal planes (parallel and perpendicular). Each of the two polarized signals is later divided into two (WL₁ and WL₂; where WL indicates wavelength) Four PMTs 11 detect and read each of the four signals at photon counting mode.

Polarization and fluorescence intensity are then calculated by a computer according to the formulae:

### Degree of Polarization

P_{WL1}=(I₁−¹−I₂−¹)/(I₁−¹+I₂−¹)

P_{WL2}=(I₁−²−I₂−²)/(I₁−²+I₂−²)

### Fluorescence Intensity

I_{WL1}=I₁−¹+I₂−¹

I_{WL2}=I₁−²+I₂−²

In this way, the system detects changes of fluorescence polarization (depolarization) and fluorescence intensity depending on biochemical conditions of the cell.

The optical system includes an excitation subsystem 100, a fluorescence subsystem 110, a projection subsystem 120 and a removable eyepiece 130 for optical calibration.

Excitation subsystem 100 includes laser light source 14, as detailed hereinabove. Subsystem 100 further includes Rotating variable ND optical filter 22 (e.g. Reynard Corp., San Clemente, USA), part #510, optical density range 0 to 1. System 100 further includes an optical shutter, pictured here as Q-switch 24 (RTP 4×4×20 (2×10) mm (Raicol Crystals, Ariel, Israel). Part #1041/42) and ND optical filter 26 (Linos, Goettingen, Germany), part #371142, τ=10%.

Other components of subsystem 100 are Lens 28 (Linos, Goettingen, Germany part #311347), dichroic mirror 30 at 45° angle (Omega optical, Brattleboro, USA part #XF2010 (505DRLP), reflection spectral range>500 nm; transmission spectral range>500 nm), Cube polarizer (Oriel, Stratford, USA part #26350), Field diaphragm 33 0.8 mm, Plate beam splitter 34 (Reynard Corp., San Clemente, USA part #880.1, τ=15%, r=85%; (where τ represents transmission and r represents reflection), Photo detector 36 (Centronic, New Addington, England), and microscope objective 40 LWD CD Plan 40^{x} dry (Olympus, Hamburg, Germany).

ND filter 22 and photo detector 36 are responsible for keeping the laser intensity at a constant level. Q-switch 24 regulates the duration of laser excitation exposure. This prevents bleaching and biological damage and allows measurement of cells which might exceed the maximum measurable excitation in systems which lack an optical shutter. This is achievable by setting a maximum number of photons and recording a time at which this maximum is reached. Cells which reach this pre-set maximum can then be ranked according to time, those having the shortest time exhibiting the strongest excitation..

ND filter 26 provides additional lowering of laser intensity. Lens 28 and microscope objective lens provide a laser spot of 18-20 microns at the cell carrier 5 plane (FIG. 1). Dichroic mirror 30 enables passing of laser excitation in one direction and fluorescence emitted from the cells in the other direction. Polarizer 32 provides a higher extinction ratio of polarization. The beam splitter 34 divides the laser excitation beam into two. Most of the energy enters the microscope objective and a small part enters photo detector 36 for accurate laser intensity measurements and regulation.

Fluorescence subsystem 110 includes microscope objective 40 which may be, for example LWD CD Plan 40× dry (Olympus, Hamburg, Germany). Objective 40 collects fluorescence radiation from the cell. Further included in subsystem 110 is plate beam splitter 34 (Reynard Corp., San Clemente, USA, part #880.; 1, τ=15%, r=85%. Beam splitter 34 directs this radiation towards field diaphragm 33. Field diaphragm 33 0.8 mm restricts the field of view, so that the system measures radiation from a single cell at a time. Cube polarizer 32 Oriel, Stratford, USA, part #26350) divides the fluorescence beam into two beams that are polarized at two orthogonal planes.

Further included in subsystem 110 is 45° dichroic mirror 30 (Omega optical, Brattleboro, USA, part#XF2010 (505DRLP)) with a reflection spectral range<500 nm and a transmission spectral range>500 nm. The two dichroic mirrors 30, 31 prevent unwanted laser reflections.

System 110 further includes flat polarizer 42 (Melles Griot, Irvine, Calif., USA, part #03 FPG 001) to increase the extinction ratio of the two polarized beams. Two beam splitters 44a, b (Linos, Goettingen, Germany part #344141,r/τ=50% / 50%; further divide each of the two polarized beams into two. Two flat mirrors 46a, b (Linos, Goettingen, Germany part #340083) aid beam splitters 44a, b in directing the four polarized fluorescent beams toward the emission filters 48a, b and 51a, b.

Subsystem 110 further includes emission filters 48a, b and 51a, b. These may be, for example two pairs of Omega optical (Brattleboro, USA) filters part #XF3022 (580DF30) and XF3007 (535DF35) or two pairs of CVI laser corporation filters (part #F10-510.0-4-1.00 and F10-510.0-4-1.00; Orlando, Fla., U.S.A.) Choice of filters 48a, b and 51a, b will depend upon the specific embodiment of scanner 50. Emission filters 48a, b and 51a, b transmit fluorescent radiation of the required wavelengths toward each of the four PMTs 11.

Projection system 120 includes illuminating halogen bulb 4 (FIG. 1) (Heine XHL, Herrsching, Germany), part #X-02.88.044. Bulb 4 illuminates cell carrier 5 (FIG. 1) on movable XYZ stage 3, 2.

Projection system 120 further includes microscope objective 40 LWD CD Plan 40× dry (Olympus, Hamburg, Germany). Objective 40 produces an image of cell carrier 5 at the reticle 52 (optical target in the objective image plane) plane (magnification up to 40×). Projection system 120 further includes plate beam splitter 35 (Reynard Corp., San Clemente, USA), part #845.1, τ=85%, r−15%. (The symbol τ indicates transmission and r indicates reflection)Plate beam splitter 35 and IR LED 53 are used for illumination of reticle 52 at the orientation of the cell carrier 5 (FIG. 1). IR LED 53 may be, for example, an LED supplied by OSRAM Opto semiconductors (Munich, Germany). Projection system 120 further includes reticle 52 (Linos, Goettingen, Germany), part #391130, which is the optical target for grid orientation.

Projection system 120 further includes lens 54 (Linos, Goettingen, Germany), part #311338. Lens 54 projects images of the cell carrier 5 and reticle 52 image to CCD camera 9.

Adjusting eyepiece 130 includes flat mirrors 46c (Linos, Goettingen, Germany), part #340083, lens 56 (Linos, Goettingen, Germany), part #311310 and lens 58 (Optosigma), part #015-0040. Flat mirror 46c directs the light beam from the field diaphragm that is illuminated by the halogen bulb toward the lenses 56 and 58. Lenses 56 and 58 together with user's eye produce the field diaphragm image. Eyepiece magnification is preferably approximately 10×.

During use of scanner 50 a laser beam passes through ND filters 22 and 26 and lens 28. The beam is subsequently reflected from dichroic mirror 30 and enters polarizer 32. The linearly polarized excitation beam, passes through the field diaphragm 33, reflected from the beam splitter 34, passes through the microscope objective 40 and illuminates one cell (that is in the center of the field of view at the time of measurement).

As the cells fluoresce, they emanate photons which are collected by the objective 40, reflected from beam splitter 34 and passed through field diaphragm 33, which restricts the field so that only one cell is read. The photons then reach the polarizer 32. Here the fluorescent beam is divided into two separate beams that are polarized in two orthogonal planes.

The first polarized fluorescent beam passes through two dichroic mirrors 30 and 31 and is further divided into two, by beam splitter 44b. This pair of beams reaches the emission filters 48b and 51b while only one of the beams is reflected from the flat mirror 46b.

The second polarized fluorescent beam that is reflected from cube polarizer 32, passes through flat polarizer 42, and is divided into another pair of beams by a second beam splitter 44a. This pair of beams also reaches the emission filters 48a and 51a. Again, only one beam is reflected from beam splitter 44a while the second is reflected from flat mirror 46a.

Orientation of the cell carrier 5 is achieved by the projection system. An image of the cell carrier 5, is projected from the objective's imaging plane (reticle's 52 plane) onto the CCD camera, by the lens 54. Magnification m=−1^{×}. (The minus sign means inverted image magnification). Simultaneously the same lens projects the image of the reticle 52, which is illuminated by IR LED 53, onto CCD camera 9. (Magnification m=−1^{×}). This causes two images to appear on video monitor 10. The first image is a movable image of the cell carrier 5 and the second image is an unmovable image of the reticle as a background.

System 65 further comprises a computerized control mechanism 15 designed and configured to co-ordinate actions of grid holder 71 containing grid 5, vacuum source 74, population of cells 81 in suspension, liquid reservoir 75, loading device 76 and electro-optical scanner 50. The at least one liquid may be applied to the individual cells either from space 72 or from upper surface 82 of grid 5. Grid holder 71 includes space 72, at least one port 73, and at least one port 73 serving for removal of liquid from space, (Port 73 is pictured as two ports 73a and 73b.

The present invention is further embodied by an automated method 84 of collection of data from a plurality of individual cells belonging to a population of cells 81 in suspension. The method includes the steps of providing 83 cell carrier grid 5 including plurality of individual discrete locations 77 arranged in an array such that each of individual locations 77 is capable of engaging and retaining one cells 81, and holding the grid in a grid holder 77 such that lower surface 80 of grid 5 is in communication with space 72 within holder 71. Method 84 further includes the step of allowing 87 at least one liquid to enter and leave space 72 in holder 71 via port 73 and the step of causing 89 the individual cells from population of cells 81 to move into the locations 77 by means of vacuum source 74 connectable to port 73. . Method 84 further includes the step of supplying the population of cells 81 in suspension and allowing 90 communication between the at least one liquid in liquid reservoir 75 and individual cells from population of cells while individual cells reside in locations 77. The at least one liquid may communicate with the individual cells either from space 72 or from upper surface 82 of grid 5. Method 84 further includes the step of employing a loading device 76 to facilitate communication between grid holder 71 containing grid 5, vacuum source 74, population of cells 81, and liquid reservoir 75. Method 84 further includes the step of illuminating 95 the individual cells residing in individual discrete locations 77 and collecting at least a portion of photons emanating therefrom by means of an electro-optical scanner 50. Method 84 further includes the step of co-ordinating actions of grid holder 71, vacuum source 74, population of cells 81, liquid reservoir 75, loading device 76 and electro-optical scanner 50 by means of a computerized control mechanism 15.

Also within the scope of the present invention is an article of manufacture useful for collection of data from a plurality of individual cells belonging to a population of cells 81 in suspension in a clinical setting. The article of manufacture includes a cell carrier grid 5 as described hereinabove held in grid holder 71 as described hereinabove. The article of manufacture further includes vacuum source 74, liquid reservoir 75, loading device 76 electro-optical scanner 50 and computerized control mechanism 15 as described hereinabove. Preferably computerized control mechanism 15 is operable with a graphical user interface. Application of vacuum via port 73 causes the individual cells from population of cells 81 to move into individual discrete locations 77.

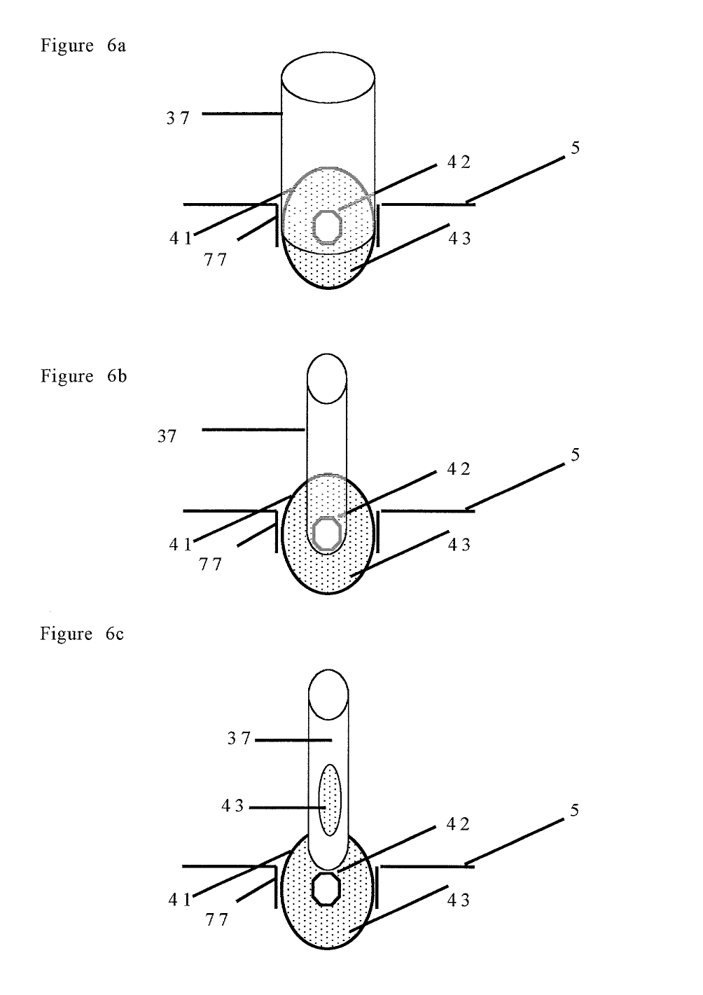

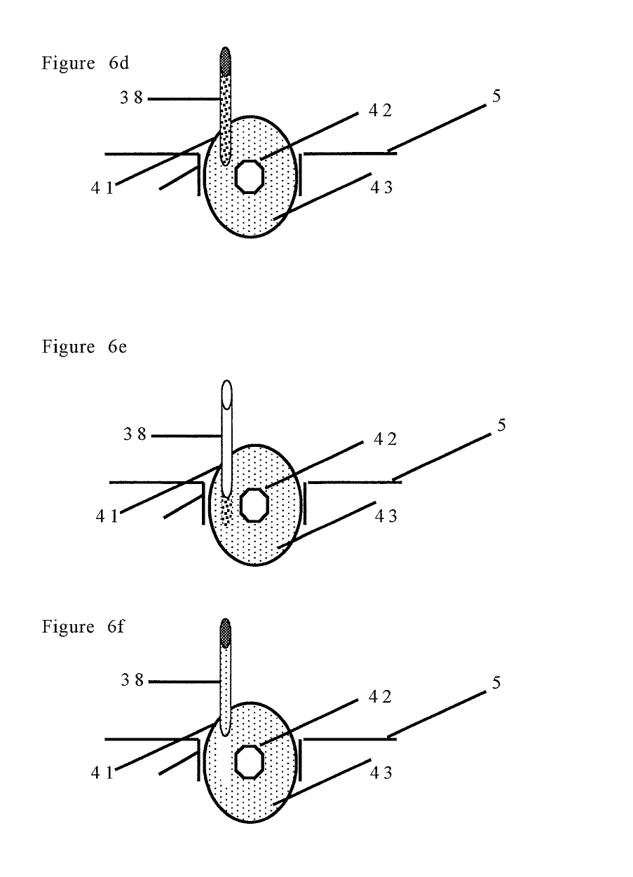

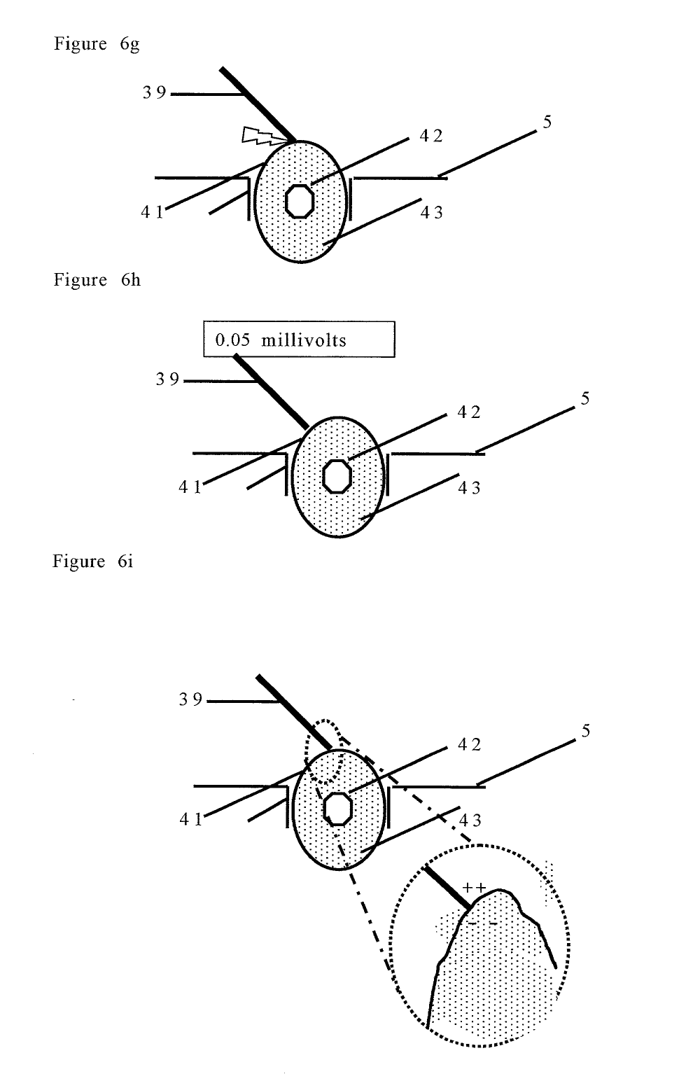

The present invention further includes an improved electro-optical scanner 50 (FIGS. 1, 2 and 3) capable of individually collecting data from a plurality of individual cells 81 residing in predefined locations 77. Scanner 50 includes an optical unit. The optical unit includes camera 9, light source 14, photomultiplier 11, optical shutter 24, and at least one optical filter 22 and 26. Scanner 50 scans a cell carrier grid 5 as described hereinabove. In order to scan grid 5 scanner 50 further includes a scanning unit capable of exposing the discrete locations of grid 5 to light from light source 14. Scanner 50 further includes a cell manipulation device 7. Cell manipulation device 7 may be, for example a micropipette 37 (FIG. 6), a needle 38, or an electrode 39. Scanner 50 further includes control unit 15 which includes a computer designed and configured for co-ordinating actions of the optical unit, grid 5, scanning unit 1, 2, and 3 and cell manipulation device 7.

The present invention further includes among its various preferred embodiments a method 84 of collecting data from individual cells belonging to a plurality of individual cells 81 residing in predefined locations 77 by means of improved electro-optical scanner 50. Method 84 includes the steps of causing individual cells from the plurality of individual cells 81 to be engaged and retained in discrete locations belonging to an array of discrete locations 77 in cell carrier grid 5 by applying force thereto 89. Method 84 further includes the step of exposing the discrete locations to light 95 from a light source 14 by employing a scanning assay unit 50 and the step of generating data from an optical unit as described hereinabove. Method 84 further includes the step of manipulating 96 individual cells from the plurality of individual cells 81 with a cell manipulation device 7 as described hereinabove. Method 84 further includes the step of co-ordinating actions of the optical unit, cell carrier grid 5 and cell manipulation device 7 and the scanning assay unit 50 from control unit 15 which includes a computer. Preferably, methods according to the present invention include the step of bringing the cells in the individual discrete locations into contact 90 with at least one liquid delivered, for example, from liquid reservoir 75. Method 84 may further include additional steps, including but not limited to, removing 91 grid holder 71 from loading device 76, transferring 92 grid holder 71 to scanning assay device 50 and removing 101 grid holder 71 from scanning assay device 50. These steps may be performed, for example, by robotic mechanism 78 designed and configured for that purpose. Preferably, the steps of automatically filling 87 space 72, and automatically applying a force 89 are accomplished by causing a liquid to flow 93 through port 73 in grid holder 71.

According to preferred embodiments of the invention, electro-optical scanner 50 includes an optical unit which includes camera 9, light source 14, photomultiplier 11, optical shutter 24, and optical filter 22 Hand 26. Electro-optical scanner 50 further includes a scanning unit 1, 2, and 3 capable of exposing discrete locations 77 to light from light source 14. The optical unit and components thereof and the scanning unit are controlled by computerized control mechanism 15 as detailed hereinabove. Electro-optical scanner 50 may further include a cell manipulation device 7 including, but not limited to, a micropipette 37, a needle 38, or an electrode 39. In such a case, control unit 15 further co-ordinates actions of cell manipulation device 7.

Micropipette 37 may be employed, for example to remove 97 at least a portion of an organelle from an individual cell. FIG. 6b illustrates removal of cell nucleus 45 from a cell after micropipette 37 has penetrated cell membrane 41. According to alternate embodiments of the invention, only genomic DNA is removed from nucleus 45. Alternately or additionally (FIG. 6c), at least a portion of the individual cell's cytoplasm 43 is removed by micropipette 37. Alternately or additionally (FIG. 6a) micropipette 37 removes the individual cell from one of discrete locations 77.

Needle 38 may be employed for injecting (FIG. 6d and 6e) a substance into an individual cell residing in discrete location 77 or extracting (FIG. 6f) a substance from an individual cell residing in discrete location 77.

Electrode 39 may be employed for, for example, applying an electric current (FIG. 6g) to an individual cell residing in discrete location 77, measuring (FIG. 6h) a potential difference across a membrane of an individual cell residing in the discrete location, or creating (FIG. 6i) a potential difference across a membrane 41 of an individual cell residing in the discrete location.

Preferably, electro-optical scanner 50 is capable of collecting at least a portion of photons emanating from the individual cells residing in individual discrete locations 77 and is further capable of gathering polarization data pertaining to the photons. According to preferred embodiments of the invention, this polarization data is useful in making a medical diagnosis.

Methods according to the present invention may include the additional step of providing at least one robotic mechanism 78. Robotic mechanism 78 may perform functions including, but not limited to, placing 86 grid holder 71 into loading device 76, removing 91 grid holder 71 from loading device 76, transferring 92 grid holder 71 to assay device 50; and removing grid holder 71 from assay device 50.

According to additional preferred embodiments of the invention, the method includes the additional step of including within the electro-optical scanner a cell manipulation device 7 as described hereinabove and further co-ordinating actions of cell manipulation device 7 by control unit 15.

Preferably the article of manufacture further includes instructions for performing specific analyses therewith, the instructions reducing the need for calibration thereof. Alternately or additionally, the article of manufacture may further include a cell manipulation device 7 as described hereinabove.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. An automated system for loading individual cells from a population of cells in suspension into individual discrete locations within an array of individual discrete locations located in a cell carrier grid contained in a cell carrier grid holder, the system comprising:
(a) the cell carrier grid, the grid held in the cell carrier grid holder such that a lower surface of the grid is in communication with a space within the holder;
(b) the cell carrier grid holder, the holder comprising:
(i) said space in communication with said lower surface of the grid;
(ii) at least one port for introduction of a liquid into said space;
(iii) said at least one port further serving for removal of said liquid from said space;
(c) a vacuum source connectable to said port;
(d) at least one liquid reservoir for bringing at least one liquid into contact with the individual cells from a population of cells in suspension while the individual cells reside in the individual discrete locations; and
(e) a loading device facilitating communication between the grid holder containing the grid, said vacuum source, the population of cells in suspension, and said at least one liquid reservoir;
wherein application of vacuum via said port causes the individual cells from the population of cells in suspension to move into the individual discrete locations; and
wherein said at least one liquid may be applied to the individual cells from a location selected from the group consisting of said space and an upper surface of the cell carrier grid.

2. The system of claim 1, wherein said grid holder is constructed of at least one material selected from the group consisting of Lucite, plastic, and glass, silicon metal.

3. The system of claim 1, further comprising at least one robotic mechanism.

4. The system of claim 3, wherein said at least one robotic mechanism is designed and configured for performing at least one function selected from the group consisting of:
(i) placing the grid holder into said loading device;
(ii) removing the grid holder from said loading device
(iii) transferring the grid holder to a scanning assay device;
(iv) removing the grid holder from said scanning assay device.

5. The system of claim 3, wherein said robotic mechanism includes at least one item selected from the group consisting of at least one robotic arm, at least one conveyor belt, at least one pneumatic tube, at least one piston and at least one rotating plate.

6. The system of claim 1, wherein said port comprises a first port serving for introduction of a liquid into said space and a second port serving for removal of said liquid from said space.

7. The system of claim 1, further comprising a computerized control mechanism designed and configured to co-ordinate the actions of said vacuum source, the at least one population of cells in suspension, said loading device and said at least one liquid reservoir.

8. The system of claim 3, further comprising a computerized control mechanism designed and configured to co-ordinate the actions of said vacuum source, the at least one population of cells in suspension, said loading device, said at least one liquid reservoir, and said at least one robotic mechanism.

9. The system of claim 1, wherein at least one reagent contained within said at least one liquid is capable of imparting a measurable degree of fluorescence to the cells in the suspension at at least one wavelength.

10. The method of claim 9, wherein said at least one reagent capable of imparting a measurable degree of fluorescence is selected from the group consisting of:
(a) a substance that differentially stains living cells;
(b) a precursor of a fluorescent substance that differentially stains living cells;
(c) a fluorophore that stains nucleic acids; and
(d) a flourescently labeled antibody.

11. An automated method for loading individual cells from a population of cells in suspension into individual discrete locations within an array of individual discrete locations located in a cell carrier grid contained in a cell carrier grid holder, the method comprising the steps of:
(a) placing the grid holder into a loading device;
(b) automatically filling a space in the cell carrier grid holder with a liquid such that said liquid fills the individual discrete locations;
(c) automatically adding a portion of the cells in suspension to an upper surface of the grid; and
(d) automatically applying a force to said portion of the cells in suspension so that individual cells enter at least some of the individual discrete locations.

12. The method of claim 11, further comprising the step of:
(e) bringing the cells in the individual discrete locations into contact with at least one liquid.

13. The method of claim 11, wherein said step of placing the grid holder into a loading device is further automated.

14. The method of claim 11, wherein said grid holder is constructed of at least one material selected from the group consisting of Lucite, plastic, glass, silicon and metal.

15. The method of claim 11, wherein said step of placing the grid holder into said loading device is accomplished with the aid of at least one robotic mechanism.

16. The method of claim 11, wherein at least one additional step selected from the group consisting of:
(i) removing the grid holder from said loading device;
(ii) transferring the grid holder to a scanning assay device; and
(iii) removing the grid holder from said scanning assay device;
is performed by said at least one robotic mechanism which is further designed and configured for performing said at least one additional step.

17. The method of claim 15, wherein said robotic mechanism includes at least one item selected from the group consisting of at least one robotic arm, at least one conveyor belt, at least one pneumatic tube, at least one piston and at least one rotating plate.

18. The method of claim 16, wherein said robotic mechanism includes at least one item selected from the group consisting of at least one robotic arm, at least one conveyor belt, at least one pneumatic tube, at least one piston and at least one rotating plate.

19. The method of claim 11, wherein said steps of automatically filling a space, and automatically applying a force are accomplished by causing a liquid to flow through at least one port in said grid holder.

20. The method of claim 19, wherein causing said liquid to flow includes causing said liquid to flow through:
(i) a first port serving for introduction of said liquid into said space; and
(ii) a second port serving for removal of said liquid from said space.

21. The method of claim 11, wherein said steps of automatically filling a space, automatically adding a portion of the cells, and automatically applying a force are co-ordinated by a computerized control mechanism.

22. The method of claim 17, wherein said steps of automatically filling a space, automatically adding a portion of the cells, and automatically applying a force are co-ordinated by a computerized control mechanism which further controls said at least one robotic mechanism.

23. The method of claim 16, wherein said steps of automatically filling a space, automatically adding a portion of the cells, and automatically applying a force are co-ordinated by a computerized control mechanism which further controls said at least one robotic mechanism.

24. The method of claim 11, wherein at least one reagent contained within said liquid is capable of imparting a measurable degree of fluorescence to the cells in the suspension at at least one wavelength.

25. The method of claim 24, wherein said at least one reagent capable of imparting a measurable degree of fluorescence is selected from the group consisting of:
(a) a substance that differentially stains living cells;
(b) a precursor of a fluorescent substance that differentially stains living cells;
(c) a fluorophore that stains nucleic acids; and
(d) a flourescently labeled antibody.

26. An automated system useful for collection of data from a plurality of individual cells belonging to a population of cells in suspension, the system comprising:
(a) a cell carrier grid including a plurality of individual discrete locations arranged in an array such that each of said individual discrete locations is capable of engaging and retaining one of the individual cells, said grid held in a grid holder such that a lower surface of the grid is in communication with a space within said holder;
(b) said cell carrier grid holder comprising:
(i) said space in communication with said lower surface of the grid;
(ii) at least one port for introduction of a liquid into said space;
(iii) said at least one port further serving for removal of said liquid from said space;
(c) a vacuum source connectable to said port;
(d) at least one liquid reservoir for bringing at least one liquid into contact with the individual cells from the population of cells in suspension while the individual cells reside in the individual discrete locations; and
(e) a loading device facilitating communication between said grid holder containing said grid, said vacuum source, the population of cells in suspension, and said at least one liquid reservoir;
wherein application of vacuum via said port causes the individual cells from the population of cells in suspension to move into the individual discrete locations; and
wherein said at least one liquid may be applied to the individual cells from a location selected from the group consisting of said space and an upper surface of the cell carrier grid;
(f) an electro-optical scanner capable of illuminating the individual cells residing in said individual discrete locations and collecting at least a portion of photons emanating from the individual cells residing in said individual discrete locations; and
(g) a computerized control mechanism designed and configured to co-ordinate actions of said cell carrier grid holder, said vacuum source, the at least one population of cells in suspension, said at least one liquid reservoir, said loading device and said electro-optical scanner.

27. The system of claim 26, wherein said electro-optical scanner comprises:
(i) an optical unit, said optical unit comprising a camera, a light source, a photomultiplier, an optical shutter, and at least one optical filter; and
(ii) a scanning unit capable of exposing said discrete locations to light from said light source;
wherein said optical unit and components thereof and said scanning unit are controlled by said computerized control mechanism.

28. The system of claim 26, wherein said grid holder is constructed of at least one material selected from the group consisting of Lucite, plastic, glass, silicon and metal.

29. The system of claim 26, further comprising at least one robotic mechanism.

30. The system of claim 29, wherein said at least one robotic mechanism is designed and configured for performing at least one function selected from the group consisting of:
(i) placing the grid holder into said loading device;
(ii) removing the grid holder from said loading device
(iii) transferring the grid holder to a scanning assay device;
(iv) removing the grid holder from said scanning assay device.

31. The system of claim 29, wherein said robotic mechanism includes at least one item selected from the group consisting of at least one robotic arm, at least one conveyor belt, at least one pneumatic tube, at least one piston and at least one rotating plate.

32. The system of claim 26, wherein said port comprises a first port serving for introduction of a liquid into said space and a second port serving for removal of said liquid from said space.

33. The system of claim 26, wherein said electro-optical scanner further comprises a cell manipulation device selected from the group consisting of a micropipette, a needle, and an electrode;
wherein said control unit further co-ordinates actions of said cell manipulation device.

34. The system of claim 33, wherein said micropipette is capable of an action selected from the group consisting of removing at least a portion of an organelle from an individual cell, removing at least a portion of the individual cell's cytoplasm, and removing the individual cell from one of said discrete locations.

35. The system of claim 33, wherein said needle is capable of an action selected from the group consisting of injecting a substance into an individual cell residing in said discrete location and extracting a substance from an individual cell residing in said discrete location.

36. The system of claim 33, wherein said electrode is capable of an action selected from the group consisting of applying an electric current to an individual cell residing in said discrete location, measuring a potential difference across a membrane of an individual cell residing in said discrete location, and creating a potential difference across a membrane of an individual cell residing in said discrete location.

37. The system of claim 26, wherein at least one reagent contained within said at least one liquid is capable of imparting a measurable degree of fluorescence to the cells in the suspension at at least one wavelength.

38. The system of claim 37, wherein said at least one reagent capable of imparting a measurable degree of fluorescence is selected from the group consisting of:
(a) a substance that differentially stains living cells;
(b) a precursor of a fluorescent substance that differentially stains living cells;
(c) a fluorophore that stains nucleic acids; and
(d) a flourescently labeled antibody.

39. The system of claim 26, wherein said electro-optical scanner capable of collecting at least a portion of photons emanating from the individual cells residing in said individual discrete locations is further capable of gathering polarization data pertaining to said photons.

40. The system of claim 39, wherein said polarization data is useful in making a medical diagnosis.

41. An automated method of collection of data from a plurality of individual cells belonging to a population of cells in suspension, the method comprising the steps of:
(a) providing a cell carrier grid including a plurality of individual discrete locations arranged in an array such that each of said individual discrete locations is capable of engaging and retaining one of the individual cells, and holding said grid held in a grid holder such that a lower surface of the grid is in communication with a space within said holder;
(b) allowing at least one liquid to enter and leave said space in said grid holder via at least one port;
(c) causing the individual cells from the population of cells in suspension to move into the individual discrete locations by means of a vacuum source connectable to said port;
(d) supplying the population of cells in suspension;
(e) allowing communication between said at least one liquid in at least one liquid reservoir and the individual cells from the population of cells in suspension while the individual cells reside in said individual discrete locations wherein said at least one liquid may communicate with the individual cells from a location selected from the group consisting of said space and an upper surface of the cell carrier grid; and
(f) employing a loading device to facilitate communication between said grid holder containing said grid, said vacuum source, the population of cells in suspension, and said at least one liquid reservoir;
(g) illuminating the individual cells residing in said individual discrete locations and collecting at least a portion of photons emanating from the individual cells residing in said individual discrete locations by means of an electro-optical scanner; and
(h) co-ordinating actions of said cell carrier grid holder, said vacuum source, the population of cells in suspension, said at least one liquid reservoir, said loading device and said electro-optical scanner by means of a computerized control mechanism.

42. The method of claim 41, wherein said electro-optical scanner comprises:
(a) an optical unit, said optical unit comprising a camera, a light source, a photomultiplier, an optical shutter, and at least one optical filter; and
(b) a scanning unit capable of exposing said discrete locations to light from said light source;
wherein said optical unit and components thereof and said scanning unit are controlled by said computerized control mechanism.

43. The method of claim 41, wherein said grid holder is constructed of at least one material selected from the group consisting of Lucite, plastic, glass, silicon and metal.

44. The method of claim 41, comprising the additional step of providing at least one robotic mechanism.

45. The method of claim 44, wherein said at least one robotic mechanism performs at least one function selected from the group consisting of:
(i) placing said grid holder into said loading device;
(ii) removing said grid holder from said loading device
(iii) transferring said grid holder to a scanning assay device;
(iv) removing said grid holder from said scanning assay device.

46. The method of claim 44, wherein said robotic mechanism includes at least one item selected from the group consisting of at least one robotic arm, at least one conveyor belt, at least one pneumatic tube, at least one piston and at least one rotating plate.

47. The method of claim 41, wherein said at least one port comprises a first port serving for introduction of a liquid into said space and a second port serving for removal of said liquid from said space.

48. The method of claim 41, comprises the additional step of including within said electro-optical scanner a cell manipulation device selected from the group consisting of a micropipette, a needle, and an electrode;
wherein said control unit further co-ordinates actions of said cell manipulation device.

49. The method of claim 48, wherein said micropipette is capable of performing at least one step selected from the group consisting of removing at least a portion of an organelle from an individual cell, removing at least a portion of the individual cell's cytoplasm, and removing the individual cell from one of said discrete locations.

50. The method of claim 48, wherein said needle is capable of performing at least one step selected from the group consisting of injecting a substance into an individual cell residing in said discrete location and extracting a substance from an individual cell residing in said discrete location.

51. The method of claim 48, wherein said electrode is capable of performing at least one step selected from the group consisting of applying an electric current to an individual cell residing in said discrete location, measuring a potential difference across a membrane of an individual cell residing in said discrete location, and creating a potential difference across a membrane of an individual cell residing in said discrete location.

52. The method of claim 41, wherein at least one reagent contained within said at least one liquid is capable of imparting a measurable degree of fluorescence to the cells in the suspension at at least one wavelength.

53. The method of claim 52, wherein said at least one reagent capable of imparting a measurable degree of fluorescence is selected from the group consisting of:
(i) a substance that differentially stains living cells;
(ii) a precursor of a fluorescent substance that differentially stains living cells;
(iii) a fluorophore that stains nucleic acids; and
(iv) a flourescently labeled antibody.

54. The method of claim 41, wherein said step of illuminating the individual cells residing in said individual discrete locations and collecting at least a portion of photons emanating from the individual cells residing in said individual discrete locations further includes gathering polarization data pertaining to said photons.

55. The system of claim 54, wherein said polarization data is useful in making a medical diagnosis.

56. An article of manufacture useful for collection of data from a plurality of individual cells belonging to a population of cells in suspension in a clinical setting, the article of manufacture comprising:
(a) a cell carrier grid including a plurality of individual discrete locations arranged in an array such that each of said individual discrete locations is capable of engaging and retaining one of the individual cells, said grid held in a grid holder such that a lower surface of the grid is in communication with a space within said holder;
(b) said cell carrier grid holder comprising:
(i) said space in communication with said lower surface of the grid;
(ii) at least one port for introduction of a liquid into said space;
liquid from said space;
(c) a vacuum source connectable to said port;
(d) at least one liquid reservoir for bringing at least one liquid into contact with the individual cells from the population of cells in suspension while the individual cells reside in the individual discrete locations; and
(e) a loading device facilitating communication between said grid holder containing said grid, said vacuum source, the population of cells in suspension, and said at least one liquid reservoir;
wherein application of vacuum via said port causes the individual cells from the population of cells in suspension to move into the individual discrete locations; and
wherein said at least one liquid may be applied to the individual cells from a location selected from the group consisting of said space and an upper surface of the cell carrier grid.
(f) an electro-optical scanner capable of illuminating the individual cells residing in said individual discrete locations and collecting at least a portion of photons emanating from the individual cells residing in said individual discrete locations; and
(g) a computerized control mechanism designed and configured to co-ordinate actions of said cell carrier grid holder, said vacuum source, the population of cells in suspension, said at least one liquid reservoir, said loading device and said electro-optical scanner, said computerized control mechanism operable with a graphical user interface.

57. The article of manufacture of claim 56, further comprising instructions for performing specific analyses therewith, said instructions reducing the need for calibration thereof.

58. The article of manufacture of claim 56, further comprising a cell manipulation device.

59. An improved electro-optical scanner capable of individually collecting data from a plurality of individual cells residing in predefined locations, the scanner comprising:
(a) an optical unit, said optical unit comprising a camera, a light source, a photomultiplier, an optical shutter, and at least one optical filter;
(b) a cell carrier grid, said grid comprising an array of discrete locations, each of said discrete locations capable of engaging and retaining a single living cell;
(c) a scanning unit capable of exposing said discrete locations to light from said light source;
(d) a cell manipulation device selected from the group consisting of a micropipette, a needle, and an electrode and
(e) a control unit, said control unit comprising a computer designed and configured for co-ordinating actions of said optical unit, said cell carrier grid, said scanning unit and said cell manipulation device.

60. The electro-optical scanner of claim 59, wherein said micropipette is capable of an action selected from the group consisting of removing at least a portion of an organelle from an individual cell, removing at least a portion of the individual cell's cytoplasm, and removing the individual cell from one of said discrete locations.

61. The electro-optical scanner of claim 59, wherein said needle is capable of an action selected from the group consisting of injecting a substance into an individual cell residing in said discrete location and extracting a substance from an individual cell residing in said discrete location.

62. The electro-optical scanner of claim 59, wherein said electrode is capable of an action selected from the group consisting of applying an electric current to an individual cell residing in said discrete location, measuring a potential difference across a membrane of an individual cell residing in said discrete location, and creating a potential difference across a membrane of an individual cell residing in said discrete location.

63. A method of collecting data from individual cells belonging to a plurality of individual cells residing in predefined locations by means of an improved electro-optical scanner, the method comprising the steps of:
(a) causing individual cells from the plurality of individual cells to be engaged and retained in discrete locations belonging to an array of discrete locations in a cell carrier grid;
(b) exposing said discrete locations to light from a light source by employing a scanning unit;
(c) generating the data from an an optical unit, said optical unit comprising a camera, said light source, a photomultiplier, an optical shutter, and at least one optical filter;
(d) manipulating individual cells from the plurality of individual cells with a cell manipulation device selected from the group consisting of a micropipette, a needle, and an electrode; and
(e) co-ordinating actions of said optical unit, said cell carrier grid and said cell manipulation device and said scanning unit from a control unit, said control unit comprising a computer.

64. The method of claim 63, wherein said micropipette is capable of performing an additional step selected from the group consisting of removing at least a portion of an organelle from the individual cell, removing at least a portion of the individual cell's cytoplasm, and removing the individual cell from one of said discrete locations.

65. The method of claim 63, wherein said needle is capable of performing an additional step selected from the group consisting of injecting a substance into an individual cell residing in said discrete location and extracting a substance from an individual cell residing in said discrete location.

66. The method of claim 63, wherein said electrode is capable performing an additional step selected from the group consisting of applying an electric current to an individual cell residing in said discrete location, measuring a potential difference across a membrane of an individual cell residing in said discrete location, and creating a potential difference across a membrane of an individual cell residing in said discrete location.
